(19) United States
(12) Patent Application Publication
(10) Pub. No.: US 2026/0289767 A1
(43) Pub. Date: Sep. 24, 2026

(54) DENTAL IMPLANT DETECTION SYSTEM

(71) Applicant: Ajman University, AJman (AE)

(72) Inventors: Khaled Assaleh, Ajman (AE); Kamran Arshad, Ajman (AE); Alexander Maniangat Luke, Ajman (AE); Sam Thomas Kuriadom, Ajman (AE); Qammer Hussain Abbasi, Ajman (AE); Muhammad Ali Imran, Ajman (AE); Adnan Qayyum, Ajman (AE); Muhammad Zakir Khan, Ajman (AE)

(73) Assignee: Ajman University, Ajman (AE)

(21) Appl. No.: 19/088,912

(22) Filed: Mar. 24, 2025

(51) Int. Cl. G06T 7/00; G06T 7/11; G16H 30/40

(52) U.S. Cl. CPC G06T 7/0012; G06T 7/11; G16H 30/40; G06T 2207/20108; G06T 2207/30036

(57) ABSTRACT

A method includes receiving two-dimensional images. The two-dimensional images are slices from three-dimensional electronic scans. The two-dimensional images are for a region of interest within a person's mouth. The method includes generating a benchmark database. The benchmark database includes views of different mouth areas. The method includes determining a dental implant with the person's mouth.

### BACKGROUND

Detection of the mandibular canal (also referred to as the Inferior Alveolar Nerve (IAN) canal) in dental images is an important task conducted in the preoperative planning of dental surgeries. In traditional dental practice, this is often done manually by dental radiologists to avoid damage to the IAN inside the mandibular canal. Various studies have proposed the use of Machine Learning (ML) and Deep Learning (DL) techniques for the automatic detection of mandibular canals. However, the practicality of these methods is hindered by various challenges like the availability of representative datasets comprising good quality images and annotations. Accordingly, there is currently no effective method to scan this particular area of the mouth using such techniques.

Dental implants provide a long-term treatment (solution) for missing teeth that can enhance a patient's oral wellness, quality of life and self-confidence. Dental implants can be used as single-tooth, partial, and total edentulism. Pre-implant planning is crucial to ensure the efficacy of the implant and to avoid any unforeseen circumstances.

In conventional dental clinical practice, panoramic and intraoral radiographs are employed in planning dental implant that provides a panoramic view of the jaws that provides a rough estimate about the implant. Nevertheless, these radiographic methods are insufficient for comprehensive implant planning. Also, the majority of dentists and oral radiologist are not well trained on analyzing the radiographic dental images and therefore, they lack competency in outlining a detailed pre-implant plan by interpretating the dental anatomical data (which is required to be extracted from dental radiographic images).

### BRIEF DESCRIPTION OF DRAWINGS

FIG. 1 is a diagram of example flow diagram;

FIGS. 2, 3, 4, and 5 are diagrams of example modules;

FIG. 6 is a diagram of an example image;

FIG. 7 is a diagram of an example image;

FIG. 8 is a diagram of an example image;

FIGS. 9, 10, and 11 are diagrams of example images;

FIG. 12 is a diagram of an example table;

FIGS. 13 and 14 are diagrams of example graphs;

FIGS. 15A, 15B, 15C, and 15D are diagrams of example images;

FIG. 16 is a diagram of an example table;

FIGS. 17, 18, 19 and 20 are diagrams of example images;

FIG. 21 is a diagram of an example table;

FIGS. 22A, 22B, 22C, and 22D are diagrams of example images;

FIGS. 23A, 23B, 23C, and 23D are diagrams of example images;

FIGS. 24A, 24B, 24C, and 24D are diagrams of example images;

FIG. 25 is a diagram of an example flow process;

FIG. 26 is a diagram of an example flow process;

FIG. 27 is a diagram of an example flow process;

FIG. 28 is a diagram of an example flow process;

FIGS. 29A and 29B are diagrams of an example flow process;

FIGS. 30A, 30B, and 30C are diagrams of an example flow process;

FIG. 31 is a diagram of an example table;

FIG. 32 is a diagram of example images;

FIGS. 33A and 33B are diagrams of an example flow process;

FIG. 34 is a diagram of an example image;

FIG. 35 is a diagram of an example table;

FIG. 36 is a diagram of an example networking environment; and

FIG. 37 is a diagram of an example computer.

### DETAILED DESCRIPTION OF PREFERRED EMBODIMENTS

The following detailed description refers to the accompanying drawings. The same reference numbers in different drawings may identify the same or similar elements.

Systems, devices, and/or methods described herein are to determine the location of the mandibular canal (also referred to as the Inferior Alveolar Nerve (IAN) canal) within a person's mouth by using two-dimensional (2D) panoramic slices extracted from three-dimensional (3D) CBCT) Cone Beam Computed Tomography) scans (also known as the Mandibular Canal Detection Dataset (MCD2).

In embodiments, a benchmark database for mandibular canal segmentation named Mandibular Canal Detection Dataset (MCD2) is generated. In embodiments, the MCD2 database contains a number of unilateral mandible slices that have been extracted from panoramic slices generated using 3D CBCT dental scans that have different views, i.e., full, medium, and small views. In embodiments, the MCD2 database provides bilateral (full view panoramic) mandible slices. Furthermore, to benchmark MCD2, different state-of-the-art neural network architectures are used and include (but not limited to) standard U-Net, Attention U-Net, DeepLabv3, and Fully Convolution Network (FCN). In addition, different backbone networks across different families of classification models with these models, namely ResNets, mobilenet, and VGG are used.

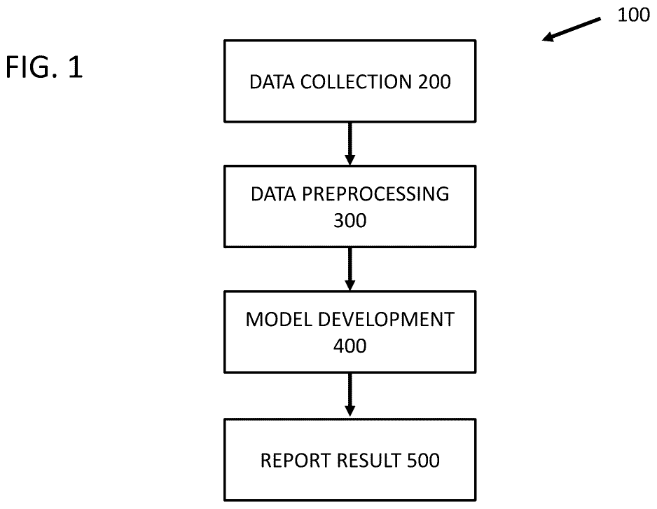

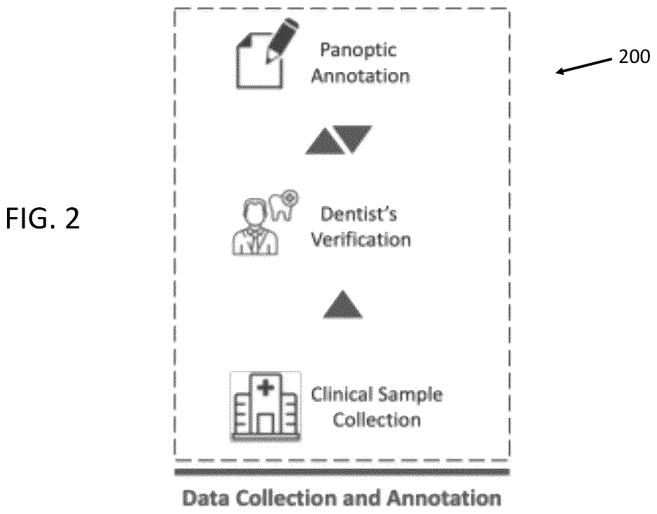

FIG. 1 describes a flowchart process 100 for obtaining electronic information and using that electronic information to determine the location of the IAN canal. As shown in FIG. 1, process 100 includes modules 200, 300, 400, and 500. In embodiments, data collection 200 includes sample collection from the clinical environment and panoptic annotation. In embodiments, the 3D CBCT images can be collected using the CS 9300 3D scanner that produces images having dimensions of 150 μm×150 μm×150 μm voxels. In embodiments, the density of the collected images may vary 49.9 μmm to 150 μmm and for better representation of the mandibular canal, the slices are sampled at the density of 1.1 μmm. FIG. 2 further describes module 200.

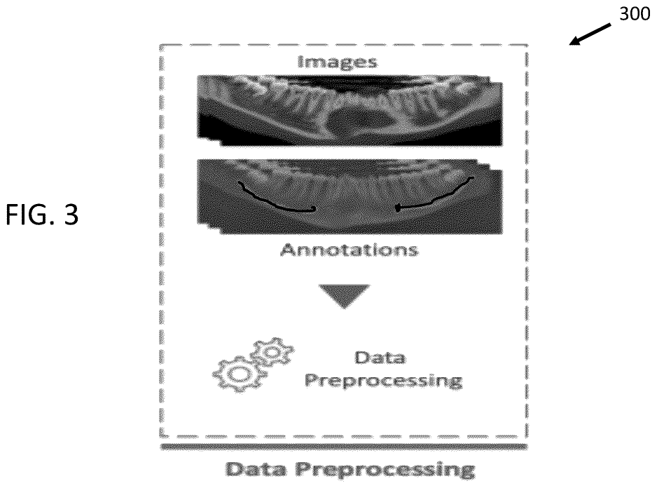

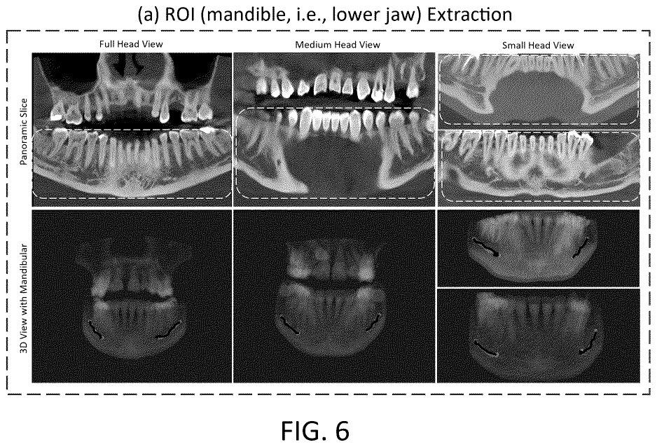

In embodiments, the 3D CBCT scans are obtained from individuals requiring dental implants in the mandible region. FIG. 3 further describes module 300 which includes a number of preprocessing steps that are applied to the data before data annotation and after data annotation. In embodiments, the collected images have different views, i.e., full head view, medium view, and small view (such as shown in FIG. 6). In embodiments, the region of interest (ROI) of the lower jaw area is extracted before performing data annotation. In embodiments, the extraction of ROI in 3D CBCT scans also avoids extra computations are be wasted in processing irrelevant pixels, which could potentially influence the learning capabilities of ML/DL models. Therefore, eliminating the irrelevant information from training data can save computations and can influence the model to learn relevant features at the same time. In addition, appropriate preprocessing is applied to remove any privacy-related information from the collected dataset.

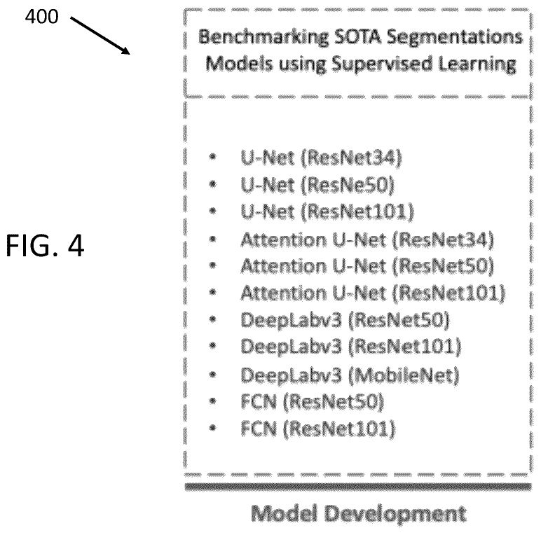

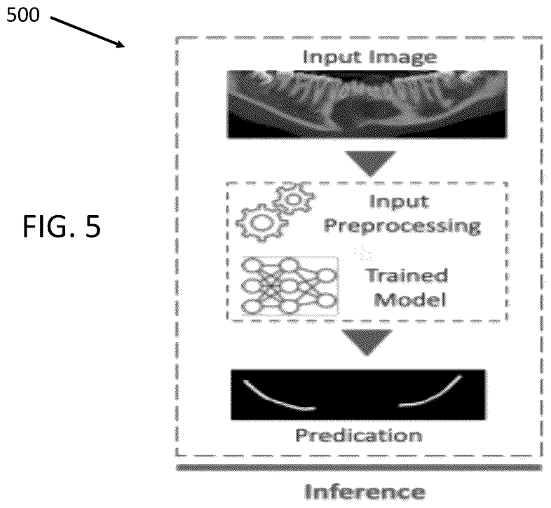

In embodiments, for efficient training of models, precise and pixel-level annotations for the identification of the mandibular canal are required. In embodiments, the data labelling strategy includes: (1) performing the annotation of collected data; (2) defining the annotation guidelines; and (3) validating and rectifying the annotations. In a non-limiting example, a set of 35 images having different quality and varied levels of difficulty in accurately detecting the path of the mandibular canal are annotated by two expert oral radiologists each having more than 20 years of experience. Then the experts trained a team of two technicians to annotate the rest of the images, which are then verified and rectified by the expert oral radiologist to ensure the efficacy of the annotations. As shown in FIG. 4, to benchmark MCD2, different state-of-the-art neural network architectures are used and include (but not limited to) standard U-Net, Attention U-Net, DeepLabv3, and Fully Convolution Network (FCN). In addition, different backbone networks across different families of classification models with these models, namely ResNets, mobilenet, and VGG are used. As shown in FIG. 5, module 500 is further shown in the end results provided in an electronically displayable report.

In embodiments, for annotation of collected 3D CBCT scans and for extracting 2D slices, CareStream (CS) 3D Imaging Software may be used, which is widely used software in dental clinical practice. This software is primarily used for analyzing 3D CBCT images and for pre-operative planning of dental implants.

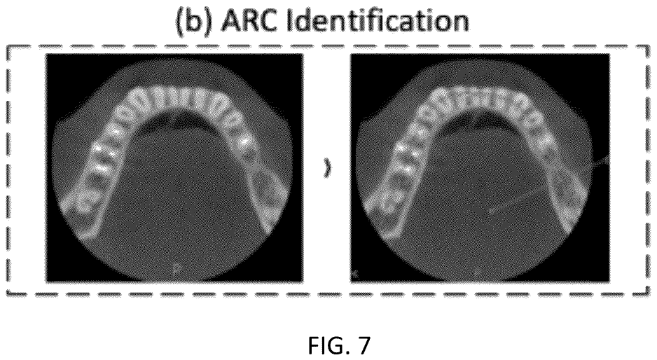

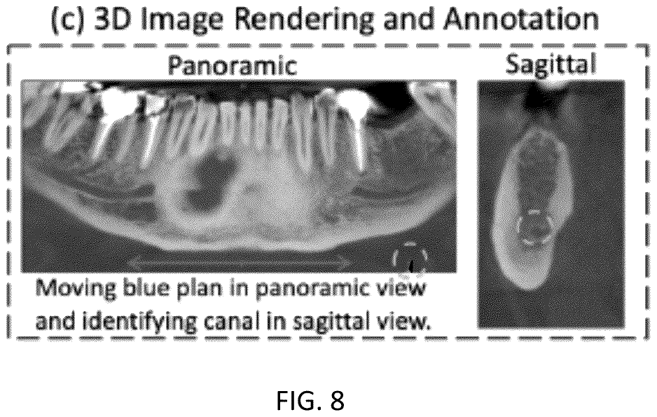

In embodiments, the methodology used for the annotation of CBCT images for the task of mandibular canal detection using CS Imaging Software is further described in FIGS. 6, 7, and 8. As shown FIG. 6, the first step is to extract ROI from the collected 3D CBCT scans. As shown in FIG. 7 ARC identification is performed by generating axial slices from the 3D CBCT scan, which are then used for the generation of respective panoramic and sagittal slices. As shown in FIG. 8, Finally, the mandibular canal path is identified by utilizing the information contained in panoramic and sagittal slices, which is performed by moving the white cursor in the panoramic slice and then identifying the canal opening in the sagittal slice. For identifying the mandibular canal at the right side, the cursor is moved from extreme right to mid until the canal closing point is seen. Similarly, it is moved from the extreme left to the point where the canal ends for the identification of the left mandibular canal.

In embodiments, the electronic data collected for process 100 is obtained from dental patients. In a non-limiting example, the dataset is collected from 89 patients who are about to undergo a dental implant. In this non-limiting example, the patients have different demographics, sex, and age. Out of 89 patients, 35 are female and 54 are male, which have different ethnicity and the ages. In this non-limiting example, the CBCT scans from 39 patients contain full mandible information, i.e., mandibular canal for both sides (left and right), while the remaining 50 patients have undergone only one-sided CBCT scans (i.e., either left or right). Therefore, in this non-limiting example, there are two sets of images of the dataset: (1) a set of 39 2D images containing bilateral mandible; and (2) an augmented set of images containing the unilateral (i.e., single-sided) mandible images including the one containing left and right mandible from full view images.

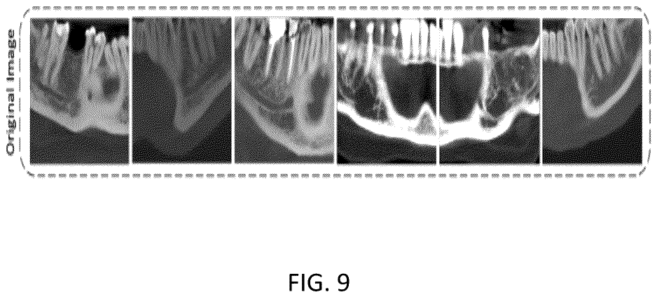

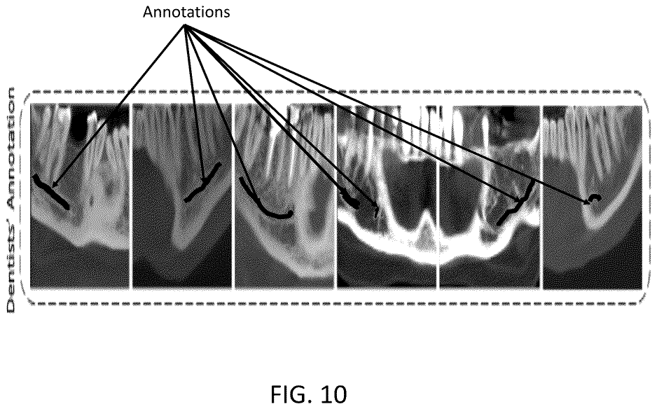

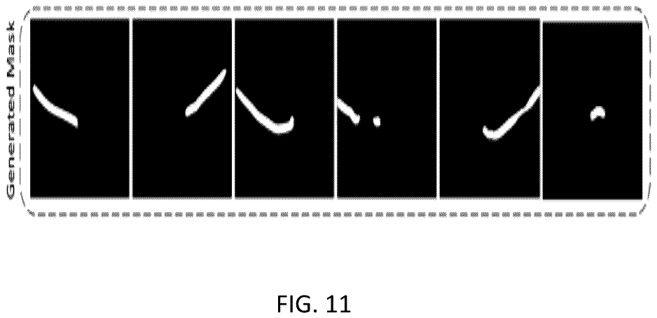

Therefore, in this non-limiting example, the final dataset has a total of 167 images. Visual examples from the collected dataset depicts different variations along with the dentists' annotations and generated masks (for model training) are shown in FIGS. 9, 10, and 11 (respectfully). In embodiments, these figures show that the collected dataset has different variations, i.e., in terms of image quality, annotation location, size, and visibility.

In embodiments, to determine mandibular canal detection, an input CBCT image is split into two distinct (non-overlapping) regions, i.e., foreground (the mandibular canal region) and background (region except mandibular canal information). In embodiments, this formulation includes a typical binary classification task at the pixel level so as to identify which class a particular pixel belongs to. In embodiments, the models are training using the annotated data which is extracted from 3D CBCT scans.

Accordingly, the systems, methods, and/or devices described herein are to learn an unknown function that could map the input image x to the target y such that ƒ: x→y. To learn this function f, SOTA (state of the art) segmentation models are trained. In embodiments, the segmentation model is denoted as Mₛ, which is trained using paired training data

$$D = {\{ {x}_{i} , {y}_{i} \}}_{i = 1}^{N} ,$$

where xᵢ, and yᵢ represent the input image and the target (ground-truth) mask, respectively. N is the total number of samples in D, where the input image is denoted as xᵢ∈R^{n×m×3} and target segmentation mask is defined as yᵢ∈{C0,1}^{n×m} which consists of an image containing pixels intensities of 1 and 0 representing foreground (mandibular canal) and background, respectively. In embodiments, where n and m are the number of rows in input images and masks. The segmentation model Mₛ is trained using standard loss used for binary classification (as defined in Eq. 1).

$$L ( {y}_{k} , {y}_{k}^{′} ) = {y}_{k} log ( {y}_{k}^{′} - ( 1 - {y}_{k} ) log ( 1 - {y}_{k}^{′} ) ) \qquad ( 1 )$$

Where, y′ₖ denotes the mask predicted by the underlying model and yₖ is the reference (ground-truth) mask.

Accordingly, the systems, methods, and/or devices described herein provide for a benchmark dataset for segmenting the mandibular canal. Therefore, segmentation models are evaluated based on an electronic learning process. For example, models are trained and tested on labeled datasets where each input image has a corresponding ground-truth label or annotation. For segmentation tasks, this label is usually a pixel-wise mask, which indicates the class of each pixel (e.g., of the object mandibular canal) and background. In embodiments, the systems, methods, and/or devices described herein are for pixel-level classification where image pixels are classified into the background (non-mandibular canal region) and foreground (mandibular canal region) using binary cross entropy loss (equation 1). In embodiments, the classification is also based on a mask generated by Mₛ with a reference mask.

For benchmarking of MCD2, four different architectures of deep generative models and six different DL-based backbone architectures are analyzed. This included, but is not limited to, generative models: U-Net (with skip connections)36, Attention U-Net (with attention mechanism and skip connections), fully convolutional network (FCN)30, and Deeplab-v331. Furthermore, different architectures of classification models are used across different family of neural networks architectures for vision applications that include: residual network-based architectures (such as, but not limited to, ResNet34, ResNet-50, and ResNet-10132), VGG-based neural networks (such as VGG11, VGG16, and VGG1933), and Mobilenet-v334. In embodiments, these classification models are integrated with the aforementioned generative models to get the benchmark results using MCD2.

In a non-limiting example, the total number of samples collected from the MCD2 dataset may 167. Also, in this non-limiting example, the data is divided into two non-overlapping sets using a split of 80% and 20% for training and testing, respectively. In embodiments, since the ROI size in the collected dataset is different, therefore, all images are resized to a uniform rectangular dimension of 210×300. In embodiments, the models are trained on large-scale datasets for similar segmentation tasks such as semantic segmentation using Microsoft's COCO dataset35. Furthermore, to ensure the efficient training of models, standard data augmentation methods are used for increasing the size of the training set.

For training of standard U-Net and Attention U-Net models (with different backbone models), a batch size of either two (bilateral mandibular images) or four (unilateral mandibular images) may be used. Also, these models are trained for a maximum of 25 epochs using a learning rate (LR) of 1e⁻³, where the optimal LR is determined using the LR scheduler, which scales weight adjustments to minimize the network's loss36. In case LR is low, the training advances at a slow pace, which happens due to minor adjustments to model weights. Conversely, a high LR can lead to undesirable divergent behavior. To facilitate model learning, a cyclic LR approach for model training can be used.

Since the purpose of this paper is to benchmark the dataset, therefore, three different widely used performance metrics for the evaluation of the models include: (1) Mean Intersection Over Union (mIoU)—also known as Jaccord similarity index; (2) Dice score—also referred to as F1-score in the evaluation of segmentation models; and (3) average pixel accuracy. Average Pixel Accuracy: The percentage of accurately classified pixels in the model's reconstructed mask is known as average pixel accuracy, mathematically it is defined as:

$$mPA = \frac{1}{N} {\sum }_{i = 1}^{N} \frac{{n}_{ii}}{{c}_{i}}$$

where, mean average pixel accuracy is represented as mPA and N refers to the total number of pixels, nᵢᵢ denotes the total number of accurately classified pixels as class i, and cᵢ is the number of pixels predicted as class j. In nᵢᵢ, i.e., actual class labels and predicted class labels are the same (i.e., it denotes true positives). Intersection Over Union (IoU): It is a widely employed in the evaluation of segmentation models, which is also known as the Jaccord similarity index.

In embodiments, IoU calculates the pixel level overlap between the reference and the predicted masks. In embodiments, the IoU measures the overlap between the actual mandibular canal (i.e., human-annotated region) and the mask generated by the model for the potential mandibular canal path. Mathematically, IoU is calculated as:

$$IoU = \frac{TP}{( TP + FP + FN )}$$

where, TP, FP, and TN denote true positive, false positive, and true negative, respectively. Also, IoU is given as:

$$IoU ( m , {m}^{′} ) = \frac{\vert m ⋂ {m}^{′} \vert}{\vert m ⋃ {m}^{′} \vert}$$

where, m and m′ represent the reference (ground truth) mask and the predicted mask. Dice Score: It is a commonly utilized similarity metric for the evaluation of segmentation methods. This metric has been widely recognized for evaluating the segmentation quality of medical images. For the binary image segmentation problem (differentiating the background and foreground), the dice score is calculated as:

$$Dice Score = \frac{2 TP}{2 TP + FP + FN}$$

To benchmark the collected MCD2 dataset for the mandibular canal segmentation task, evaluated different SOTA models are evaluated. For example, there may be four different models across different families of neural architectures that have been proposed for the modeling of similar segmentation problems. In addition, different architectures of classification models are analyzed when integrated with mainstream segmentation models (also referred to as generative models) and analyzed the effect of neural network architectural choice on the performance of mandibular canal segmentation. Furthermore, baseline experiments can be categorized into two dimensions based on the segmentation models, i.e., using U-Net-based architectures and using other models (for comparative analysis).

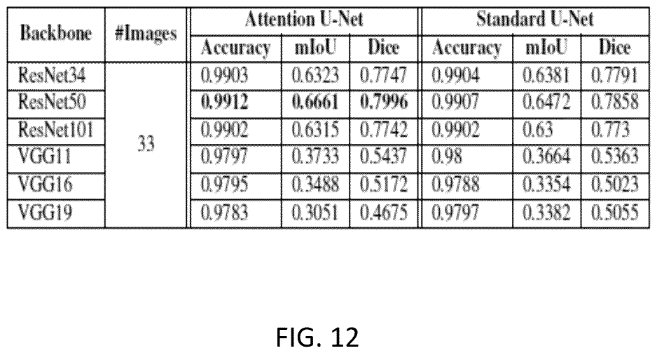

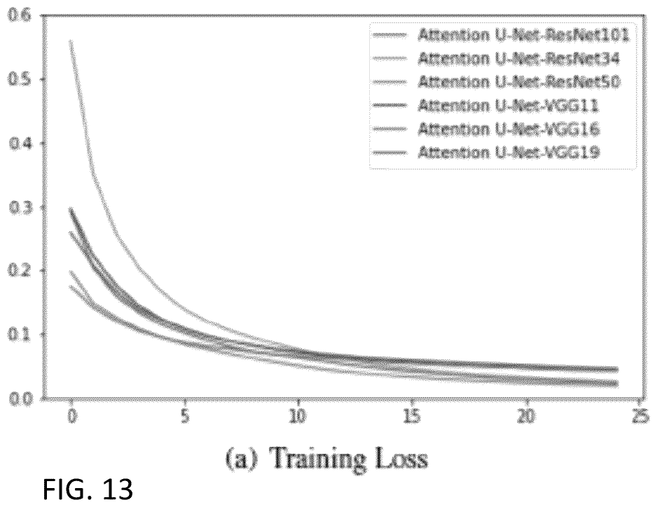

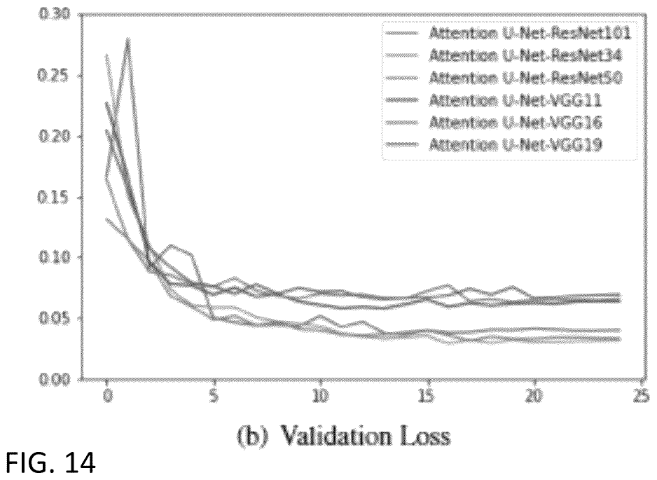

For benchmarking of MCD2, standard U-Net and Attention U-Net models as baseline models can be used. In addition, U-Net-based models are widely employed for the benchmarking of medical image segmentation datasets40. The baseline results for standard U-Net and Attention U-Net models when integrated with different backbone classification models are presented in table 1200 shown in FIG. 12. Table 1200 shows that Attention U-Net has provided relatively improved performance for mandibular canal segmentation in terms of different performance measures. Moreover, Attention U-Net with ResNet50 backbone outperform other models. In FIG. 4, learning curves (FIGS. 13 and 14, respectively) are shown for training and validation loss with increasing epochs for demonstrating the effectiveness of MCD2.

FIGS. 13 and 14 describe the baseline models smooth learning capabilities using the proposed MCD2. Moreover, FIGS. 13 and 14 show that Attention U-Net-ResNet50 depicted smoother learning as compared to other models. To further highlight the effectiveness of the U-Net with ResNet50 backbone in the detection of the mandibular canal path, visual examples using images depicting different variations in terms of image quality, mandibular canal size, shape, and location (as shown in FIGS. 15A-15D) are used. FIGS. 15A-15D highlight that U-Net having ResNet50 backbone is able to generate such masks that are very close to the human-generated masks.

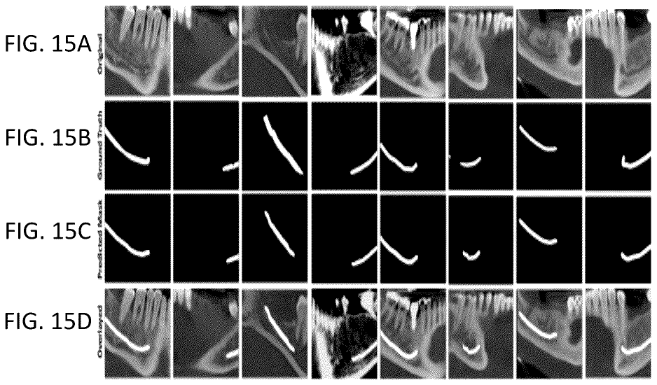

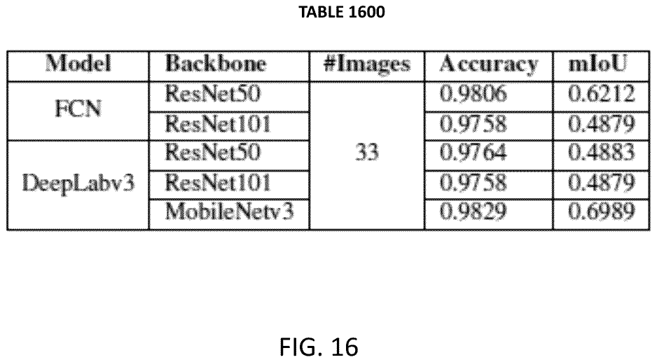

In addition, to evaluating the performance of U-Net-based models that are considered standard in benchmarking of medical image segmentation approaches. In embodiments, different SOTA models are evaluated that include: DeepLabv3 and FCN while using different backbone models. The results for this analysis are presented in table 1600 in FIG. 16, the performance of these SOTA models is not impressive in terms of different performance metrics.

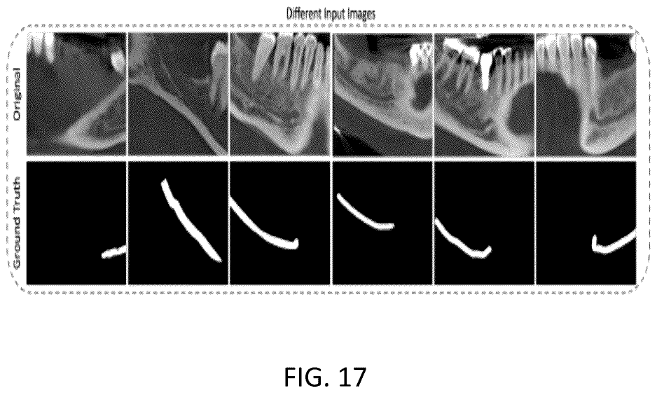

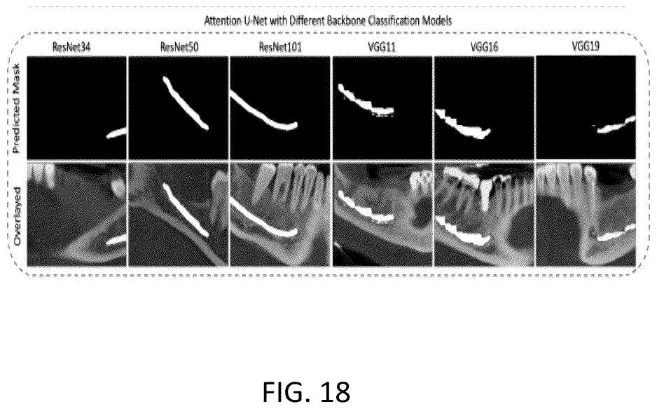

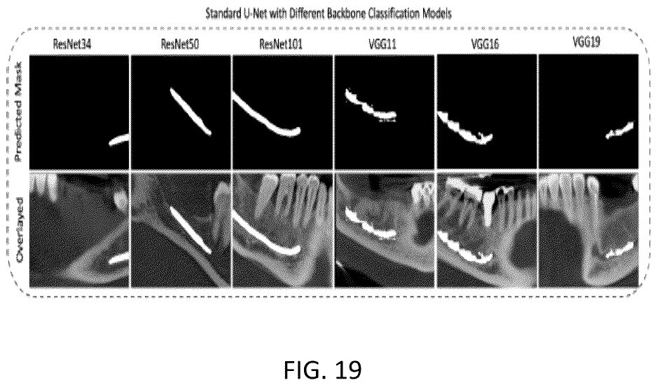

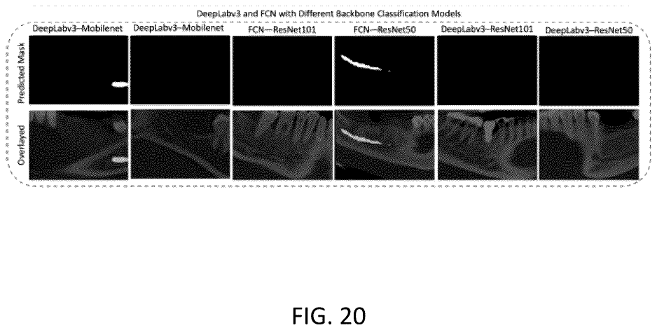

In embodiments, Table 1600 also shows that DeepLabv3 with MobileNetv3 backbone provided outperformed other models while FCN with ResNet50 backbone performance is second best across all sets of models. In embodiments, these models have very deep architecture and due to this reason, they are more prone to overfitting. On the other hand, Attention U-Net outperformed all other models including standard U-Net, DeepLabv3, and FCN-based neural network architectures, which highlights the effectiveness of incorporating attention mechanism into standard U-Net architecture (compare Table 2 and Table 3). A visual comparison of using different architectures for mandibular canal segmentation can be seen in FIGS. 17 to 20.

FIGS. 17 to 20 demonstrates that on average U-Net-based neural network architectures provided better performance as compared to other models. Also, the performance of using ResNet-based architectures as the backbone network is better as compared to using VGG-based models. ResNet-based models produce such predictions that are very close to human annotations, while VGG-based models introduce visual artifacts (as they mainly produce predictions in square shape. In embodiments, models having mIoU less than 0.5 in Table 3, i.e., DeepLabv3-ResNet50, DeepLabv3-ResNet101, and FCN-ResNet101 failed to produce predictions for the mandibular canal in most cases (see blank predicted masks in FIGS. 17-20).

As described above, MCD2 contains two types of images, i.e., unilateral mandible and bilateral mandible slices. Therefore, bilateral mandibular images (i.e., containing right and left mandible) are used for the task of jointly identifying the mandibular canal on both sides. Based on the performance of Attention U-Net in efficiently identifying the mandibular canal in unilateral slices, the same model architecture is used for this analysis. In addition, for the sake of comparison, the performance of the standard U-Net model is evaluated with different backbone architectures for the reports results for using different backbone classification models with standard U-Net and Attention U-Net in terms of different performance metrics such as mIoU, average pixel accuracy, and average dice score.

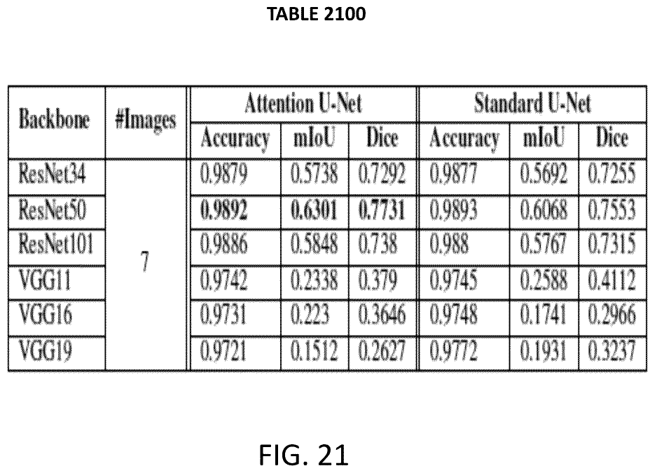

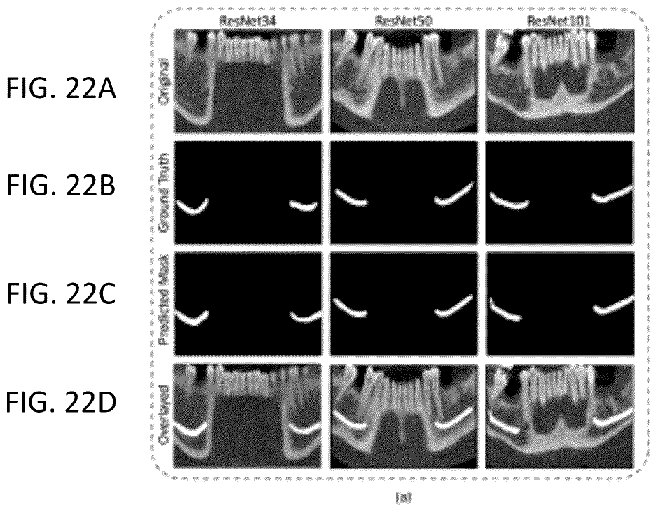

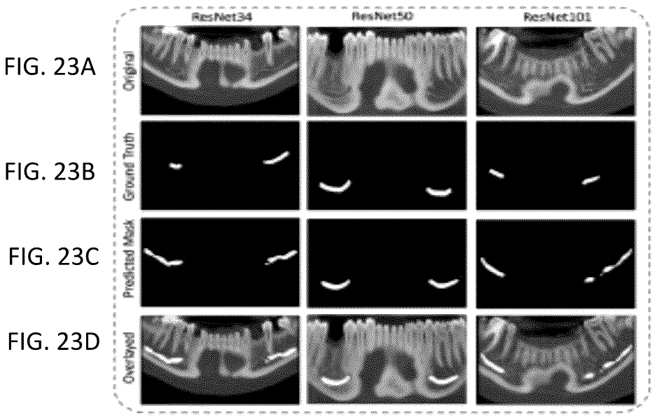

As shown in table 2100 (FIG. 21), Attention U-Net with ResNet50 backbone is shown to provide higher performance as compared to other models. A similar observation is noted in the case of using unilateral mandible images. In embodiments, the overall attention-based U-Net architectures provided improved performance as compared to the standard U-Net (with attention) model, which highlights the effectiveness of using Attention U-Net for mandibular canal segmentation. Visual results for using bilateral mandible images are presented in FIGS. 22A-22D, 23A-23D, and 24A-24D which shown detection of mandibular canals in bilateral images.

The results for these experiments are summarized in table 2100, the table reports results for using different backbone classification models with standard U-Net and Attention U-Net in terms of different performance metrics such as mIoU, average pixel accuracy, and average dice score. The table highlights that Attention U-Net with ResNet50 backbone provided higher performance as compared to other models. A similar observation is noted in the case of using unilateral mandible images. Also, the overall attention-based U-Net architectures improved performance as compared to the standard U-Net (with attention) model, which highlights the effectiveness of using Attention U-Net for mandibular canal segmentation. Visual results for using bilateral mandible images are shown in FIGS. 22A-22D, FIGS. 23A-23D, and FIGS. 24A-24D.

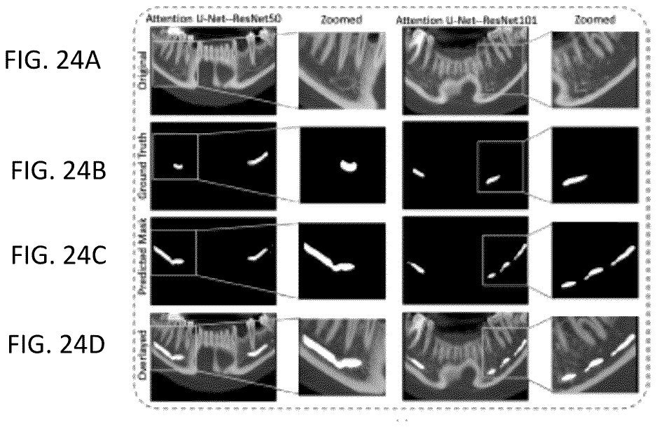

In embodiments, the models are trained using bilateral mandible images seem to outperform human annotations in most of the input images while effectively highlighting the potential path of the mandibular canal which is shown in FIGS. 24A-24D. For instance, zoom-in patches can be seen to assess the efficacy of the underlying models in producing candidate predictions for the potential path of the mandibular canal.

Systems, methods, and/or devices described herein provide for a low-cost tool based on ML/DL that can provide useful information required for implant planning of missing tooth by detecting the path of the mandibular canal and tracking the proximity to the roots of adjacent teeth. In embodiments, the systems, methods, and/or devices described herein provide for a significant improvement in the quality of treatment provided by general dentists. Additionally, the proposed systems can serve as a cutting-edge training tool for dentistry medical students, offering insight into dental structure and positively impacting overall dental training. In embodiments, the systems, methods, and/or devices described herein demonstrate the necessity of state-of-the-art AI-based systems for diagnosis and treatment planning in challenging interventional procedures in dental care.

In embodiments, the systems, methods, and/or devices described herein provide an automated system that can measure the implant parameters (i.e., the distance between the tooth root and the mandibular canal). In embodiments, segmentation is performed of a whole teeth jaw in order to identify each tooth, this knowledge together with the mandibular information can then be used for the development of complete system that would inform implant measurements automatically.

Accordingly, the systems, methods, and/or devices described herein provided for (a) collecting CBCT scans of edentulous teeth regions from a large sample of patients; (b) develop an AI algorithm to analyze CBCT scans and detect implant measurements accurately; (c) test the accuracy of the AI algorithm by comparing the results with manual measurements taken by experienced Oral Radiologist; (e) evaluate the feasibility of implementing the AI system in clinical practice; and (f) assess the clinical outcomes of using the AI system in implantology, including the success rate of implants and patient satisfaction. By achieving these objectives, the systems, methods, and/or devices described herein provide for the development of an AI-based system that can enhance the accuracy and efficacy of implant planning and placement in edentulous teeth regions.

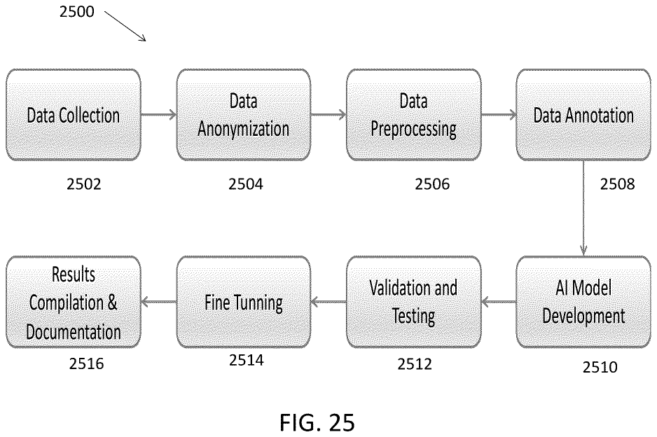

FIG. 25 is an example flowchart 2500. In embodiments, flowchart 2500 may use electronic information from patients' cases. In embodiments, flowchart 2500 is conducted by a computing device. At step 2502, appropriate data anonymization techniques are used to remove any privacy related information from the acquired images before using them for the analysis and system development. At step 2504, data preprocessing techniques is used to ensure the effectiveness and quality of the data. In embodiments, image enhancement would be used to highlight certain elements of the images, including enhancing contrast and color to highlight their best features.

At step 2508, annotation of the data is conducted with CS 3D dental imaging software. In embodiments, the distance between the tooth root and mandibular canal is annotated along with the location of the mandibular canal will be verified and validated by an oral and maxillofacial radiologist. Accordingly, images validated by the expert will be included for the analysis.

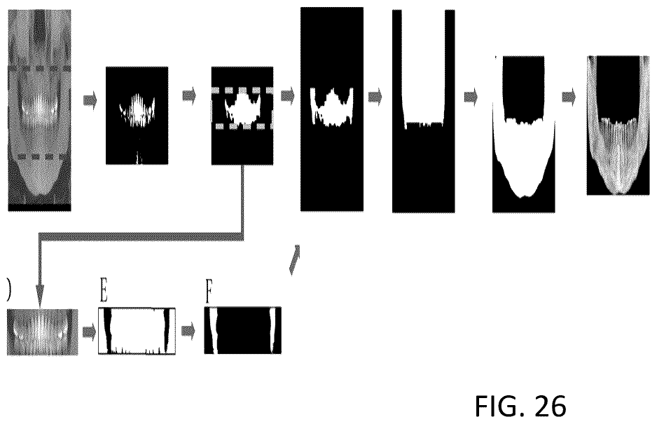

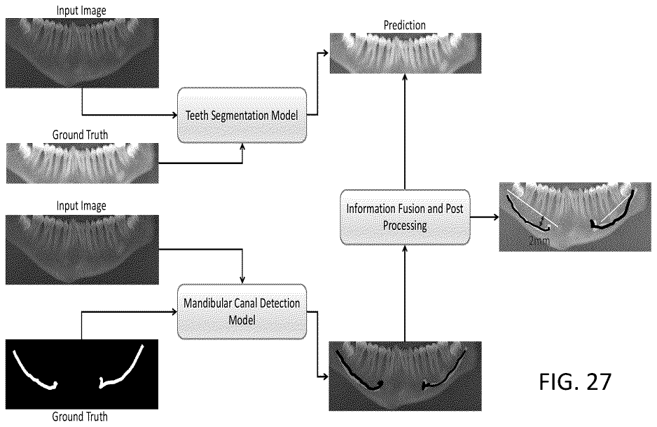

At step 2510, the annotated data is divided into training and testing set using a split of 80:20, respectively. FIG. 26 is an example preprocessing steps for extraction of lower jaw and mandibular canal detection. In embodiments, the training set will be used to train the ML/DL model, while the testing set will be used to evaluate the model's performance on unseen data. FIG. 27 shows an example framework.

As shown in FIG. 27, segmentation of teeth in the lower jaw and mandibular canal is conducted. In embodiments, the computing device uses the radiographic images as input and automatically categorizes the teeth root and mandibular canal region in the image at an expert level. This includes three components: (1) mandibular canal detection; (2) teeth segmentation; and (3) calculating measurements.

At step 2512, the computing device evaluates the developed model using test data to see its performance on the unseen data that would help in the identification of shortcomings and limitations. At step 2514, The identified limitations will be resolved by fine tuning the model and then final testing will be carried.

Accordingly, electronic data is collected for training AI models. Additionally, data annotation and preprocessing (as shown in FIG. 26) is performed to support supervised learning, which involves extracting 2D slices from 3D CBCT images and identifying key oral landmarks, such as the mandibular canal and maxillary sinus. These regions are critical in dental implants, as any damage to them could lead to serious medical complications. The systems, methods, and/or devices described herein provide for an annotation strategy which (i) for the lower jaw, the mandibular canal and measured the distance are identified between teeth and this region; and (ii) for the upper jaw, detection of the maxillary sinus and measuring the proximity of the teeth to this area. Accordingly, this comprehensive approach led to the creation of a novel; customized dataset optimized for training AI models. In embodiments, an ensemble model is developed comprising multiple components.

In embodiments, for the lower jaw, the first model is a segmentation model designed for mandibular canal segmentation, and the second model focuses on teeth segmentation and identifying missing teeth in this region. Similarly, for the upper jaw, the first model is responsible for maxillary sinus detection, and the second model handles teeth segmentation and missing teeth identification.

Once the outputs from these models are generated, a pixel-level image processing technique is applied to the combined results to estimate the distance between the root of the missing tooth and the mandibular canal in the lower jaw or the maxillary sinus in the upper jaw. For the lower jaw, the target distance is typically 2 mm above the mandibular canal, while for the upper jaw, it is 2 mm below the maxillary sinus.

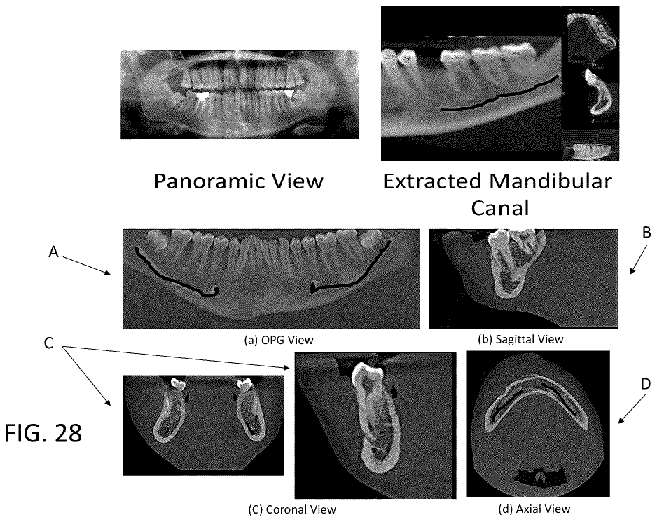

FIG. 28 is an example diagram which describes identification (tracking) of mandibular canal in panoramic radiographic finding of interruption of mandibular canal wall by third molar root. B, C, and D represents 2D slices extracted from 3-dimensional CBCT images (sagittal, coronal, and axial planes) of the same tooth indicating a direct contact between the two structures (arrows).

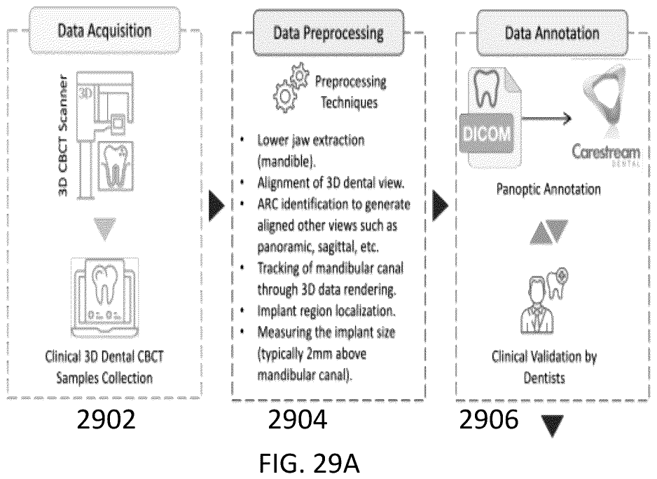

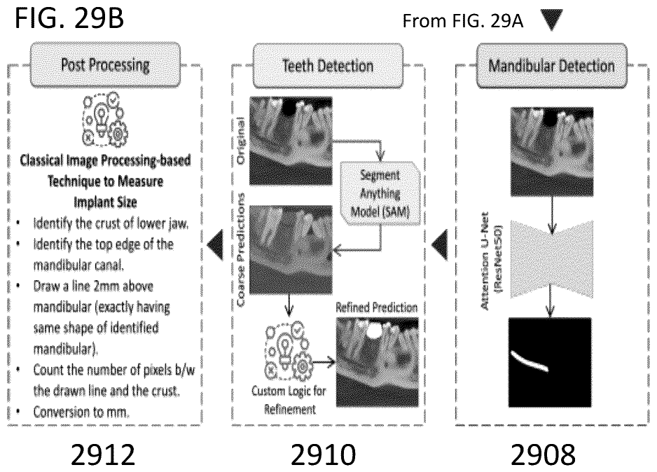

FIGS. 29A and 29B describe the process for determining implant measurements. FIGS. 29A and 29B describe 1) data acquisition; (2) data preprocessing; (3) data annotation; (4) mandibular canal detection; (5) teeth detection; and (6) post processing.

In dental implant procedures, particularly within the lower jaw, a significant challenge is the risk of damaging the mandibular nerve. This nerve is crucial, as it provides sensory input to the lower lip and chin. Damage to this nerve during implant placement can result in severe consequences, including temporary or permanent paralysis, numbness, or pain—conditions known as nerve injury or paresthesia. Therefore, the precise placement of dental implants relative to the mandibular nerve is critical to avoid such complications. However, the problem arises from the need to ensure that the implant does not breach the safety margin above the mandibular nerve. This safety margin is typically maintained at a minimum of 2 mm from the top edge of the jawbone, a measure that is crucial not only for the structural integrity of the bone but also for safeguarding against potential nerve damage.

To address this challenge, the use of 3D Cone Beam Computed Tomography (CBCT) scans is instrumental in clinical practice. As these scans provide detailed imagery that allows clinicians to visualize the mandibular nerve's exact pathway, facilitating precise implant placement. However, reliance on manual measurements and the interpretation of CBCT scans can introduce human error and variability in implant placement results. To address these challenges, there is a pressing need for the development of automated, AI-based tools. These tools could revolutionize the field by providing more consistent and accurate measurements for implant placement. We formulated the task of determining optimal implant placement as a precise measurement problem, where the goal is to accurately calculate the required implant dimensions and their correct positioning within the jawbone. Our primary objective is to derive a function f that maps an input CBCT slice x to a predicted output y that specifies implant dimensions, i.e., f:x→y. To accomplish this, training of ML/DL models is conducted under fully supervised settings using annotated CBCT scans that detail exact implant requirements.

In embodiments, the prediction model is defined as Mp, which is trained using a dataset

$$D = xi , {yi}_{i = 1}^{N} ,$$

where xᵢ represents the input CBCT image and yᵢ denotes the corresponding annotations for implant dimensions. Here, N is the total number of training samples, with each input image xᵢ∈R^{n×m×3} and the associated output yᵢ detailing the implant specifications. The annotations in yᵢ are crafted to reflect precise measurements required for optimal implant placement, such as the distance from the mandibular canal and the dimensions of the implant relative to the anatomical landmarks in the jawbone.

At step 2902, a scanner is used to capture 3D CBCT dental scans of patients requiring dental implants. The employed CBCT scanner produced 3D images with voxel dimensions of 150 μm×150 μm×150 μm, with a varying density ranging from 49.9 μm to 150 μm. To enhance the visualization of the mandibular canal and other anatomical structures, such as the roots of teeth, slices were extracted at a density of 1.1 μm. These 3D CBCT scans were performed on patients requiring dental implants in the mandible (i.e., lower jaw).

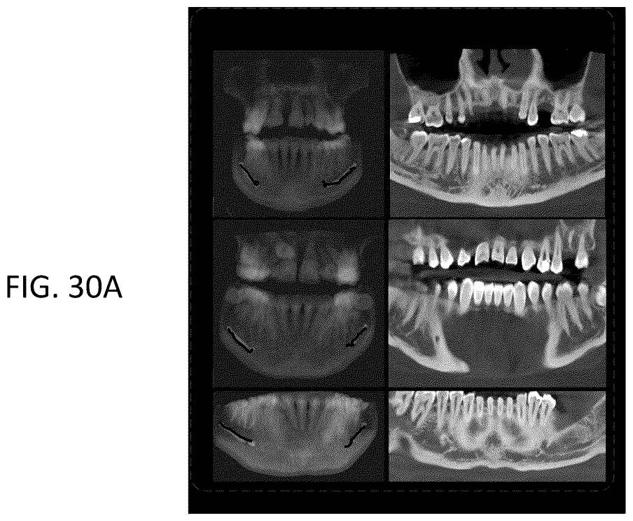

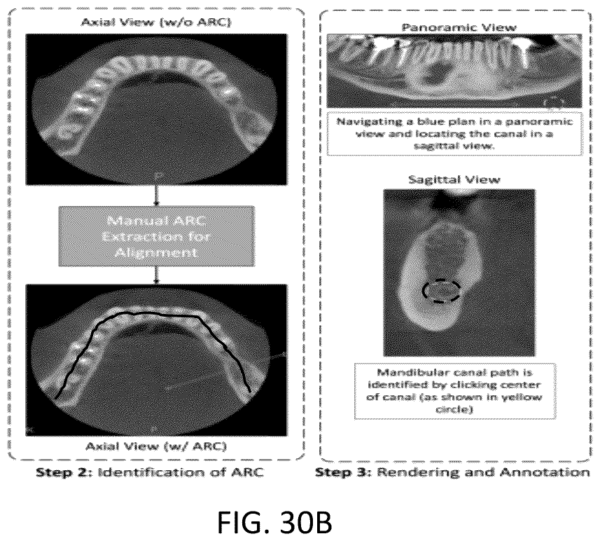

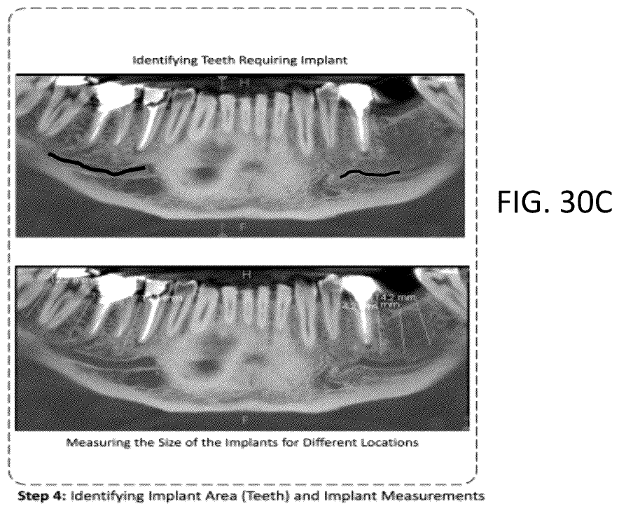

At step 2904, the data collected for this study underwent several preprocessing steps both before and after the annotations. The collected images included different scales of detail, namely full-mouth view scans, medium-mouth view scans, and closeup mouth views of specific areas such as the mandible (such as step 1, shown in FIG. 30A). Since the focus is the mandible region (i.e., the lower jaw), therefore, we initially extract this region of interest (ROI) before initiating any kind of labelling. This step is crucial not only for focusing on the relevant clinical area but also for reducing computational overhead by avoiding the processing of unnecessary pixels.

This is significant because processing irrelevant pixels can impair the efficiency of ML/DL models in exploiting critical features during training. If left unaddressed, the presence of such redundant details can potentially lead to the development of models that are not robust. Eliminating non-essential information from the training set saves computational resources and encourages the model to learn from the most relevant features.

In embodiments, at step 2906, 3D Imaging Software is used for annotating the collected 3D Cone Beam Computed Tomography (CBCT) scans. In embodiments, this software is essential for analyzing 3D images and facilitates pre-operative planning for dental implants, offering precise tools for detailed visualization and manipulation of complex dental structures. In embodiments, the annotation consists of a three-stage process carefully designed to ensure high-quality and accurate annotations. In a non-limiting example, a set of 20 images are annotated that varied in quality and difficulty level in accurately detecting the path of the mandibular canal and calculating the implant measurements. At step 2908, mandibular detection occurs. In embodiments, this includes identify the anterior region curve (ARC) for alignment purposes in the axial view of the CBCT scans. At step 2910, specific teeth are identified that require implant and measure the appropriate sizes for the implants at the specified locations within the mandible. In embodiments, these measurements are carefully calculated to ensure optimal implant placement, which is critical for the success of the surgical procedure. In embodiments, these specific measurements are taken between the crest of the jawbone and the top edge of the mandibular canal, which typically extends about 2 mm above the mandibular canal top edge. At step 2912, post processing occurs. In embodiments, this includes identifying the crust of the lower jaw, identify the top edge of the mandibular canal, generate a line that is about 2 millimeters (mm) above the mandibular canal, and determine the number of pixels between the line and the crust of the lower jaw. The calculated pixel count for the drawn line is then converted into to millimeters by multiplying it with a conversion factor based on the density of the slice's resolution.

In embodiments, the annotation process is shown in FIGS. 30A, 30B, and 30C. Following ROI extraction, the Anterior Region Curve (ARC) is identified for alignment purposes in the axial view of the CBCT scans. This procedure aids in aligning the images accurately for further processing, as illustrated in the ‘Axial View (w/ARC)’ segment in the Step 3 of in FIGS. 30A, 30B, and 30C. The ARC is shown with a dashed red line, indicating precise manual tracing. In the subsequent step, the panoramic and sagittal views are sued to locate the mandibular canal, a critical step before final annotations. This identification is vital for safeguarding critical anatomical features during the dental implant procedure. The panoramic view assists in understanding the spatial layout, while the sagittal view helps pinpoint the exact location of the canal, as shown with a yellow circle in FIGS. 30A, 30B, and 30C.

As shown in FIG. 30C, we calculate the detailed measurements for implant planning. We identify the specific teeth requiring implants and measure the appropriate sizes for the implants at the specified locations within the mandible. These measurements are carefully calculated to ensure optimal implant placement, which is critical for the success of the surgical procedure. These specific measurements are taken between the crest of the jawbone and the top edge of the mandibular canal, which typically extends about 2 mm above the mandibular top edge. In embodiments, this structured approach not only streamlines the preprocessing of CBCT scans but also ensures that every step—from the extraction of the ROI to the final measurements for implants—is conducted with the utmost precision to facilitate the development of robust ML/DL models.

In a non-limiting example, data is collected from 89 patients scheduled for dental implant surgery, representing a diverse range of demographics, including gender and age. Specifically, in this non-limiting example, the cohort consisted of 35 females and 54 males of various ethnicities. Additionally, CBCT scans from 39 patients included comprehensive mandibular data, featuring the mandibular canal on both the left and right sides. In contrast, scans from the remaining 50 patients were limited to a single side of the mandible (either left or right).

Consequently, the dataset is divided into two subsets: (1) 39 2D images with bilateral mandibular views, and (2) an expanded set of images featuring unilateral mandibular views, which also includes single-sided views extracted from full mandible scans, resulting in a total of 167 images. For the current study, such patients are identified that have missing teeth and have elderly age. Then, this subset of patients is annotated for implant measurement task.

In embodiments, the accurate detection of the mandibular canal is crucial for successful dental implant placement, as it directly influences the surgical risk management and outcome quality. To achieve high precision in mandibular canal detection, two models are evaluated that include standard U-Net and the attention U-Net architectures. In addition, to perform comprehensive evaluations, U-Net is integrated with various backbone classifiers. This strategic testing aimed to determine the most competent model for accurate segmentation of the mandibular canal and to subsequently incorporate this model into the advanced framework for dental implant planning.

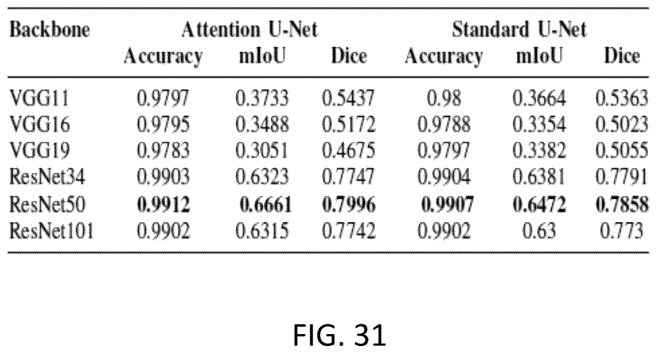

FIG. 31 is a table that summarizes the performances of various models in mandibular canal segmentation. It is evident from the table that, the attention U-Net generally outperformed the standard U-Net, especially when integrated backbone architectures such as ResNet50. In particular, attention U-Net with ResNet50 backbone achieved an accuracy of 0.9912, mIoU of 0.6661, and a Dice score of 0.7996, indicating superior segmentation capabilities. The superior performance of the ResNet50 backbone in both U-Net architectures suggests that deeper networks with residual connections are particularly effective in handling the complexities of anatomical structures in dental images. This effectiveness is likely due to their ability to retain essential information at various network depths, which is crucial for detailed feature extraction required in precise medical imaging tasks.

The comparison between the standard U-Net and attention U-Net models reveals the significant impact of attention mechanisms in enhancing model sensitivity to relevant features in medical imaging tasks. This is particularly evident in the improvement of mIoU and Dice scores, which are critical indicators of segmentation quality in medical image segmentation. Given the exceptional performance and reliability exhibited by the attention U-Net model equipped with the ResNet50 backbone, we have selected this model configuration as the foundation of our proposed framework for dental implant planning. The superior segmentation capabilities of this model, specifically in identifying the mandibular canal with high precision, strongly motivate its integration into our framework.

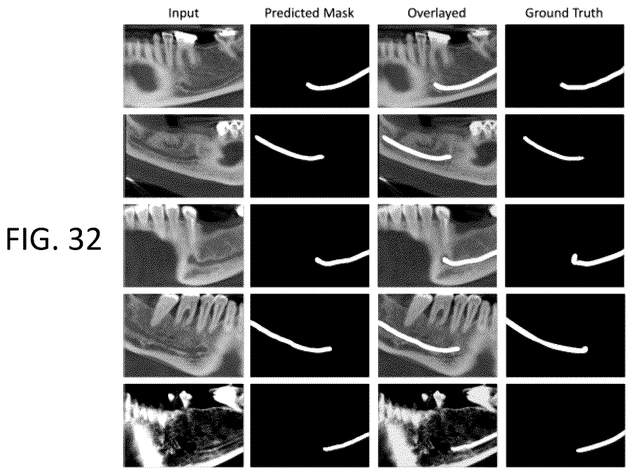

FIG. 32 describes the visual results for mandibular canal segmentation using attention U-Net with ResNet50 backbone. The predictive accuracy of the model shows the predicted masks closely matching the ground truth across various examples. Moreover, the overlayed images demonstrate how the predicted segmentation aligns precisely with the actual mandibular canal positions in the input images, even in cases with varying image clarity and contrast. These results not only validate the effectiveness of the Attention UNet with ResNet50 in capturing the intricate details necessary for accurate mandibular canal detection but also substantiate the model's robustness across different scenarios and image qualities. These outcomes further support the selection of this model for integration into our dental implant planning framework.

Accurate segmentation of teeth is a critical step in dental image analysis, particularly for implant planning and restorative procedures. The segmentation process enables the precise identification of individual teeth, allowing for comprehensive assessments of tooth structure, alignment, and missing teeth detection. The predictions from a segment anything model (SAM) are subsequently refined using a custom-designed algorithm to enhance segmentation accuracy and facilitate the detection of missing teeth.

In embodiments, the SAM is a state-of-the-art vision foundation model is employed as a first-pass segmentation tool due to its capability to generate generalized segmentations across diverse image domains. Given its ability to provide pixel-level segmentation with minimal supervision, SAM serves as an effective starting point for identifying dental structures from 2D unilateral panoramic slices extracted from 3D CBCT images. However, the coarse nature of its output necessitates further refinement to meet the precision.

To enhance the accuracy of the SAM-generated segmentations, a custom post-processing algorithm designed to refine tooth boundaries and ensure the correct identification of individual teeth. This algorithm integrates morphological operations, edge enhancement techniques, and adaptive thresholding to identify tooth structures more precisely. Additionally, missing teeth detection is performed by analyzing the refined segmentation maps, comparing them against anatomical priors, and identifying discontinuities in the expected tooth alignment. This step is essential for implant planning, as it aids in localizing the precise regions requiring dental intervention.

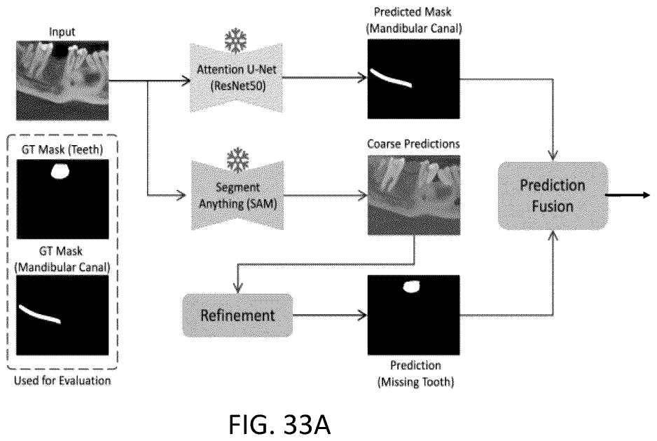

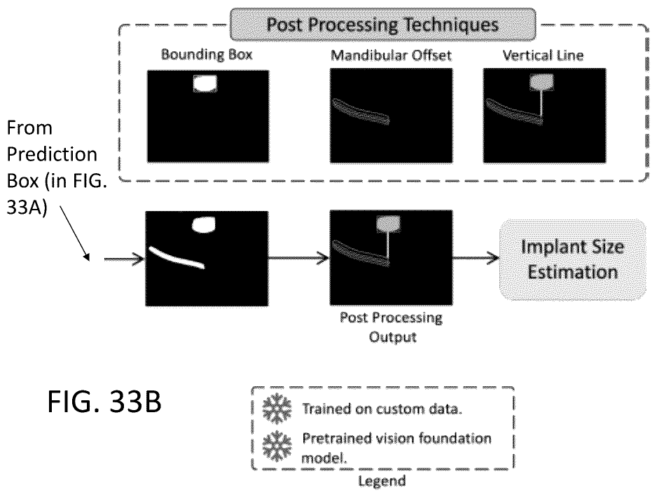

Accurate implant measurement is crucial for ensuring the success of dental implant procedures, particularly in preserving the mandibular nerve and optimizing implant placement. To achieve precise and automated implant measurement, we designed a structured pipeline that integrates segmentation, geometric analysis, and measurement computations. The proposed strategy for estimating implant measurements is shown in FIGS. 33A and 33B and described below: 1) Identification of Anatomical Structures: The first step in our framework involves the identification of the mandibular canal and missing tooth region using DLbased segmentation models. The mandibular canal is segmented to establish the critical boundary that must be preserved to avoid nerve damage, while the missing tooth region is detected to determine the implant site.

In embodiments, Attention U-Net with ResNet50, which demonstrated superior performance in mandibular canal segmentation, as discussed in the previous section. 2) Bounding Box and Central Axis Extraction: Once the missing tooth region is identified, a bounding box is generated that precisely encloses the tooth gap, ensuring it touches the contours of the missing tooth mask from all sides. In embodiments, this bounding box serves as a reference for further computations. The central axis of the bounding box is then extracted by computing its horizontal midpoint. This axis provides the vertical alignment needed for implant measurement. 3) Offset Mandibular Canal Estimation: To maintain a safety margin required during implant placement, we compute an offset mandibular canal line that follows the top contour of the canal but is positioned 2 mm above the original canal.

This offset line represents the minimum safe boundary beyond which the implant should not extend to prevent mandibular nerve damage. 4) Implant Measurement Calculation: To determine the required implant length, we draw a vertical reference line starting from the center of the bottom edge of the bounding box and extending downward until it touches the top edge of the offset mandibular canal. This vertical line represents the maximum allowable implant depth while maintaining the necessary safety margin from the nerve. The length of this vertical line is computed in millimeters using the known pixel-to-millimeter ratio, which is derived from the dental scan resolution. In our implementation, we use a conversion factor of 0.2 mm per pixel.

The dataset used in this study, comprises unilateral scans of lower jaw (which have been sourced from our previous study on mandibular canal detection). This is systematically split these scans into training (80%) and testing (20%) subsets, resulting in a particular number of images for training and another set of images for testing. Given the varying Region of Interest (ROI) sizes, all images were rescaled to a uniform resolution of 210×300 pixels to ensure standardized input dimensions. To enhance dataset diversity and improve model generalization, we incorporated standard data augmentation techniques during training, which include random rotation and horizontal flipping. These augmentation techniques were specifically applied to the mandibular canal segmentation model, which utilized the Attention U-Net architecture.

For training the Attention U-Net model, we utilized a batch size of 4 for unilateral mandibular images to ensure efficient memory utilization while maintaining stable gradient updates. The model was trained for a total of 25 epochs, allowing sufficient iterations for learning meaningful features while preventing overfitting. The learning rate was set to 1e−3 and dynamically adjusted using a cyclic learning rate (CLR) approach, as proposed by Smith [33]. This approach helps achieve optimal convergence by periodically varying the learning rate, enabling the model to escape local minima and improve generalization. For SAM, segmentation masks were automatically extracted and refined post-processing techniques were applied for missing tooth detection.

To achieve robust and precise segmentation, we adopted a multi-model fusion approach, integrating: Attention U-Net (ResNet50 backbone) for mandibular canal segmentation; and Segment Anything Model (SAM) ViT-H for teeth segmentation, including missing tooth identification. This strategy enabled the model to utilize previously learned semantic segmentation features, improving generalization. For teeth segmentation, SAM (ViT-H model) is incorporated, which was initialized using Meta's pretrained checkpoint. The SAM model was implemented using the Segment Anything API, with the automatic mask generator producing instance masks that were refined for missing tooth detection.

In embodiments, the systems, methods, and/or devices may occur in a Python environment, with DL implementations utilizing PyTorch and FastAI. Additionally, multiple supporting libraries are integrated, including the Segment Anything API for advanced segmentation, OpenCV for image preprocessing, Supervision for annotation and post-processing, and Matplotlib for visualization.

Performance Evaluation 1) Evaluating Segmentation Models: To evaluate the performance of segmentation models on the tasks of mandibular canal and teeth segmentation, we utilized three widely recognized metrics that are pertinent to the challenges of medical image segmentation. These metrics include: (1) Mean Intersection Over Union (mIoU), also known as the Jaccard similarity index; (2) Dice Score, often referred to as the F1-score in segmentation model evaluations; and (3) Average Pixel Accuracy. Average Pixel Accuracy:

This metric quantifies the proportion of pixels correctly classified by the segmentation model within the mask it reconstructs. Average Pixel Accuracy is mathematically defined as:

$$mPA = \frac{1}{N} {\sum }_{i = 1}^{N} \frac{{n}_{ii}}{{c}_{i}} ,$$

where mPA represents the mean average pixel accuracy, N is the total number of classes, nii denotes the number of pixels correctly predicted for class i, and ci is the total number of pixels classified into class i. Here, nii indicates true positives within each class. Intersection Over Union (IoU) which also referred to as the Jaccard similarity index and it measures the pixel-level overlap between the reference and the predicted masks. It is particularly useful for evaluating how accurately the model captures the area of interest, such as the mandibular canal, against the annotated regions:

$$IoU = \frac{m ⋂ \hat{m}}{\vert m ⋃ \hat{m} \vert} ,$$

where m represents the reference (ground truth) mask, and \hat{m} is the predicted mask. The IoU provides a clear measure of overlap, with higher values indicating better model performance. Dice Score:

This score is a measure of the similarity between two sets, making it highly relevant for binary segmentation tasks where the goal is to differentiate between foreground (target structure) and background. The score is calculated as follows:

$$Dice Score = \frac{2 \times TP}{2 \times TP + FP + FN} ,$$

where TP, FP, and FN denote the true positives, false positives, and false negatives, respectively. This metric is particularly relevant in medical image segmentation tasks due to its sensitivity to the size of the structures being segmented, ensuring both the presence and the precise shape of the anatomical features are captured accurately. 2) Evaluating Implant Measurements: To evaluate the performance of our proposed technique to predict dental implant measurements, we used metrics that are specifically tailored to gauge the precision and accuracy of continuous numerical predictions. These metrics are instrumental in determining how closely the predicted implant measurements align with the actual implant measurements.

Specifically, we used the following two metrics. Mean Absolute Error (MAE): This metric assesses the average magnitude of errors in predictions, without considering their direction. It's particularly useful for evaluating the precision of implant dimensions which is

$$M A E = \frac{1}{N} {\sum }_{i = 1}^{N} \vert {y}_{i} - {\hat{y}}_{l} \vert$$

where yᵢ are the true values, ŷₗ are the predicted values, and N is the number of observations. Lower MAE values indicate higher accuracy in predictions. Root Mean Square Error (RMSE): RMSE is used to measure the average magnitude of the error by squaring the differences before averaging, which gives higher weight to large errors and is sensitive to outliers:

$$R M S E = \sqrt{\frac{1}{N} {\sum }_{i = 1}^{N} {( {y}_{i} - {\hat{y}}_{l} )}^{2}}$$

This metric helps highlight larger errors in the measurement predictions.

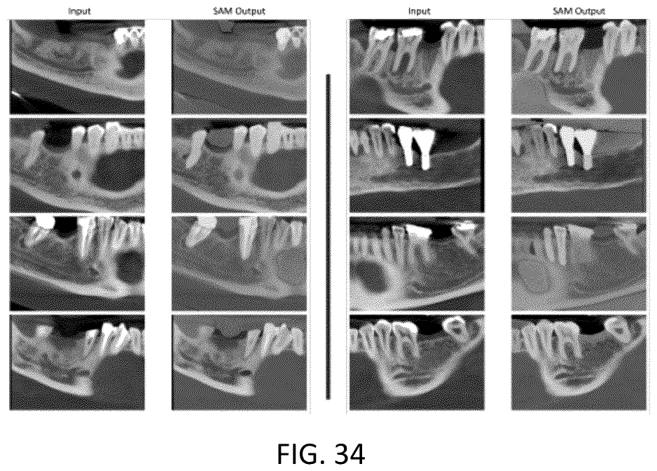

The performance of the teeth segmentation approach is assessed using multiple evaluation metrics, including Intersection Over Union (IoU), Dice score, and precision-recall analysis. These metrics quantify segmentation accuracy and validate the effectiveness of our refinement process. Ground truth annotations provided by expert dental radiologists are used for benchmarking, ensuring that the model's outputs align with clinically relevant expectations. FIG. 34 shows examples segmentation predictions generated by SAM before refinement. This example highlights the necessity of additional processing to achieve high-quality segmentations suitable for dental applications. By combining the adaptability of SAM with a customized refinement algorithm, we ensure a robust and accurate teeth segmentation framework that significantly enhances dental imaging analysis.

Following the segmentation of the mandibular canal and missing tooth region, we applied post-processing techniques to extract key implant measurement parameters. These included: (a) bounding box extraction—ensuring the mask tightly encloses the missing tooth region; (b) central axis computation—drawing a vertical line from the bounding box center to the mandibular offset; (c) mandibular canal offset calculation—generating a secondary offset canal 2 mm above the detected canal to maintain a safety margin; and (d) vertical implant measurement which includes measuring the vertical distance from the missing tooth region to the offset canal in millimeters.

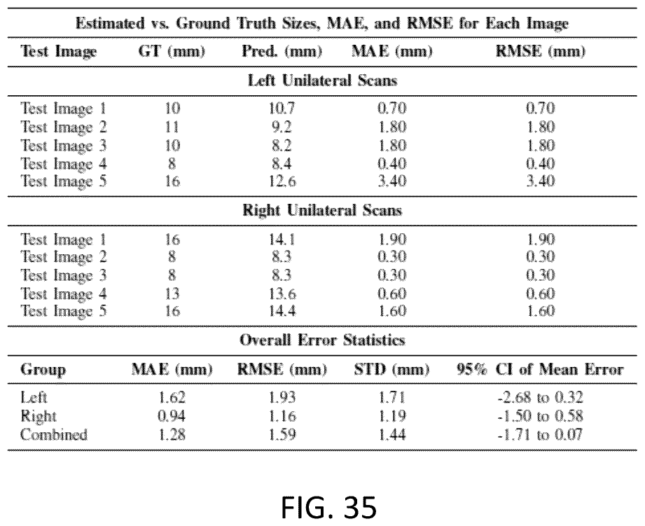

The vertical implant measurement was computed using: Lₘₘ=Lₚₓ×R_{px→mm} where Lₘₘ is the implant length in millimeters, Lₚₓ is the measured length of the vertical reference line in pixels, R_{px→mm} is the pixel-to-millimeter conversion factor determined based on the CBCT scan resolution. For our experimental setup, we used a conversion factor of 0.2 mm per pixel, leading to: Lₘₘ=Lₚₓ×0.2. The results are summarized in FIG. 35.

As shown in FIG. 35, the table summarizes the predicted implant sizes, the corresponding ground truth values, and the associated errors for both left and right unilateral scans. In addition, it includes general error statistics to evaluate the performance of the method in varying difficulty of test cases. For instance, it can be seen that for the left unilateral scans, the predicted implant sizes exhibit varying levels of deviation from the ground truth values. The lowest error is observed in Test Image 4, where the MAE and RMSE are both 0.40 mm, indicating a close match between the predicted and actual values. However, Test Image 3 and Test Image 5 show relatively larger discrepancies, with MAE values of 1.80 mm and 2.60 mm, respectively.

The highest RMSE of 3.40 mm in Test Image 5 suggests greater variability in prediction accuracy for a challenging case. On the counter side, for the right unilateral scans, the results demonstrate more consistent and accurate predictions. The errors in the five test images are generally low as compared to the predictions of lef unilateral scans. One can observe from the FIG. 35 that the lowest MAE and RMSE values (0.30 mm) are recorded in Test Images 2 and 3, reflecting high prediction accuracy. In contrast, the highest MAE value of 1.90 mm occurs in Test Image 1, which still remains within an acceptable error range.

The overall error analysis provides a broader perspective on the model's reliability. The unilateral left scans exhibit an average MAE of 1.62 mm and an RMSE of 1.93 mm, with a standard deviation (STD) of 1.71 mm, indicating greater variability in predictions for this group. The 95% confidence interval (CI) of the mean error for this group ranges from −2.68 to 0.32 mm, suggesting a wider range of potential deviations. In contrast, the right unilateral scans show lower error rates, with an MAE of 0.94 mm and an RMSE of 1.16 mm. The STD is also lower (1.19 mm), representing greater consistency in the predictions. The 95% CI of the mean error ranges from −1.50 to 0.58 mm, indicating a narrower spread of errors and improved reliability.

When combining both groups, overall performance improves, with an MAE of 1.28 mm, an RMSE of 1.59 mm, and a standard deviation of 1.44 mm. The 95% CI of the mean error (−1.71 to 0.07 mm) further provides evidence of the generalizability of the proposed metho, as the predicted values remain closely aligned with the ground truth in different test cases. The findings indicate that the proposed implant measurement estimation method achieves a reasonable level of accuracy, particularly in right unilateral scans where the errors remain consistently low. The slightly higher error rates observed in left unilateral scans suggest potential variations in anatomical structures or imaging conditions that may influence prediction performance. The overall results suggest that the model effectively estimates implant sizes with a mean error generally within 2 mm, which is clinically acceptable for preimplant planning.

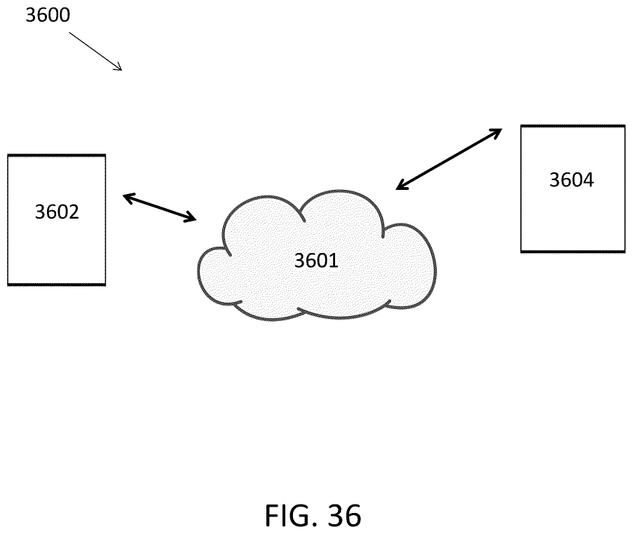

FIG. 36 is a diagram of example environment 3600 in which systems, devices, and/or methods described herein may be implemented. FIG. 36 shows network 3601, device 3602, and analysis system 3604.

Network 3601 may include a local area network (LAN), wide area network (WAN), a metropolitan network (MAN), a telephone network (e.g., the Public Switched Telephone Network (PSTN)), a Wireless Local Area Networking (WLAN), a WiFi, a hotspot, a Light fidelity (LiFi), a Worldwide Interoperability for Microware Access (WiMax), an ad hoc network, an intranet, the Internet, a satellite network, a GPS network, a fiber optic-based network, and/or combination of these or other types of networks. Additionally, or alternatively, network 3601 may include a cellular network, a public land mobile network (PLMN), a second generation (2G) network, a third generation (3G) network, a fourth generation (4G) network, a fifth generation (5G) network, and/or another network.

In embodiments, network 3601 may allow for devices describe any of the described figures to electronically communicate (e.g., using emails, electronic signals, URL links, web links, electronic bits, fiber optic signals, wireless signals, wired signals, etc.) with each other so as to send and receive various types of electronic communications.

Device 3602 may may include any computation or communications device that is capable of communicating with a network (e.g., network 3601). For example, device 3602 may include X-ray machine, a dental imagery machine, a smart phone, a desktop computer, a laptop computer, or another type of computation or communications device.

Device 3602 may receive and/or display content. The content may include objects, data, images, audio, video, text, files, and/or links to files accessible via one or more networks. Content may include a media stream, which may refer to a stream of content that includes video content (e.g., a video stream), audio content (e.g., an audio stream), and/or textual content (e.g., a textual stream). In embodiments, an electronic application may use an electronic graphical user interface to display content and/or information via device 3602. Device 3602 may have a touch screen and/or a keyboard that allows a user to electronically interact with an electronic application. In embodiments, a user may swipe, press, or touch device 3602 in such a manner that one or more electronic actions will be initiated by device 3602 via an electronic application. User device 3602 may receive electronic information from analysis system 3606 and generate and display graphs such as those described in the figures above.

Device 3602 may include a variety of applications, such as, for example, an e-mail application, a telephone application, a camera application, a video application, a multi-media application, a music player application, a visual voice mail application, a contacts application, a data organizer application, a calendar application, an instant messaging application, a texting application, a web browsing application, a blogging application, and/or other types of applications (e.g., a word processing application, a spreadsheet application, etc.). In embodiments, user device 3602 may be used to generate graphs (such as those described in previous figures) to model various features determined by analysis system 3606.

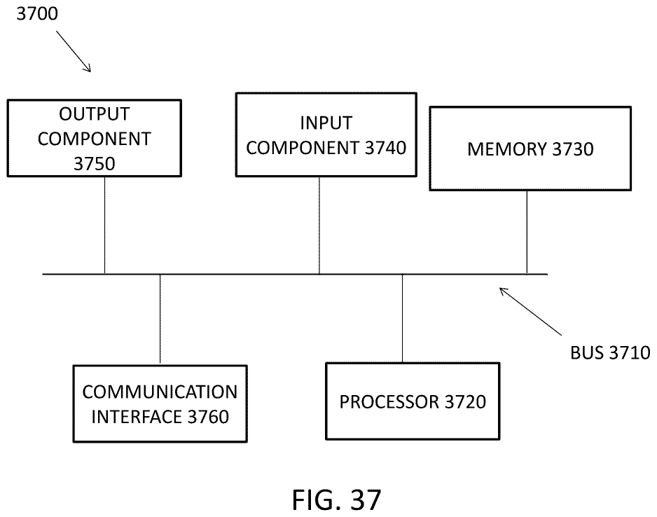

FIG. 37 is a diagram of example components of a device 3700. Device 3700 may correspond to device 3702 and analysis system 3706. Alternatively, or additionally, device 3702 and analysis system 3706 may include one or more devices 3700 and/or one or more components of device 3700.

As shown in FIG. 37, device 3700 may include a bus 3710, a processor 3720, a memory 3730, an input component 3740, an output component 3750, and a communications interface 3760. In other implementations, device 3700 may contain fewer components, additional components, different components, or differently arranged components than depicted in FIG. 37. Additionally, or alternatively, one or more components of device 3700 may perform one or more tasks described as being performed by one or more other components of device 3700.

Bus 3710 may include a path that permits communications among the components of device 3700. Processor 3720 may include one or more processors, microprocessors, or processing logic (e.g., a field programmable gate array (FPGA) or an application specific integrated circuit (ASIC)) that interprets and executes instructions. Memory 3730 may include any type of dynamic storage device that stores information and instructions, for execution by processor 3720, and/or any type of non-volatile storage device that stores information for use by processor 3720. Input component 3740 may include a mechanism that permits a user to input information to device 3700, such as a keyboard, a keypad, a button, a switch, voice command, etc. Output component 3750 may include a mechanism that outputs information to the user, such as a display, a speaker, one or more light emitting diodes (LEDs), etc.

Communications interface 3760 may include any transceiver-like mechanism that enables device 3700 to communicate with other devices and/or systems. For example, communications interface 3760 may include an Ethernet interface, an optical interface, a coaxial interface, a wireless interface, or the like.

In another implementation, communications interface 3760 may include, for example, a transmitter that may convert baseband signals from processor 3720 to radio frequency (RF) signals and/or a receiver that may convert RF signals to baseband signals. Alternatively, communications interface 3760 may include a transceiver to perform functions of both a transmitter and a receiver of wireless communications (e.g., radio frequency, infrared, visual optics, etc.), wired communications (e.g., conductive wire, twisted pair cable, coaxial cable, transmission line, fiber optic cable, waveguide, etc.), or a combination of wireless and wired communications.

Communications interface 3760 may connect to an antenna assembly (not shown in FIG. 37) for transmission and/or reception of the RF signals. The antenna assembly may include one or more antennas to transmit and/or receive RF signals over the air. The antenna assembly may, for example, receive RF signals from communications interface 3760 and transmit the RF signals over the air, and receive RF signals over the air and provide the RF signals to communications interface 3760. In one implementation, for example, communications interface 3760 may communicate with network 3601.

As will be described in detail below, device 3700 may perform certain operations. Device 3700 may perform these operations in response to processor 3720 executing software instructions (e.g., computer program(s)) contained in a computer-readable medium, such as memory 3730, a secondary storage device (e.g., hard disk, CD-ROM, etc.), or other forms of RAM or ROM. A computer-readable medium may be defined as a non-transitory memory device. A memory device may include space within a single physical memory device or spread across multiple physical memory devices. The software instructions may be read into memory 3730 from another computer-readable medium or from another device. The software instructions contained in memory 3730 may cause processor 3720 to perform processes described herein. Alternatively, hardwired circuitry may be used in place of or in combination with software instructions to implement processes described herein. Thus, implementations described herein are not limited to any specific combination of hardware circuitry and software.

It will be apparent that example aspects, as described above, may be implemented in many different forms of software, firmware, and hardware in the implementations illustrated in the figures. The actual software code or specialized control hardware used to implement these aspects should not be construed as limiting. Thus, the operation and behavior of the aspects are described without reference to the specific software code—it being understood that software and control hardware could be designed to implement the aspects based on the description herein.

Even though particular combinations of features are recited in the claims and/or disclosed in the specification, these combinations are not intended to limit the disclosure of the possible implementations. In fact, many of these features may be combined in ways not specifically recited in the claims and/or disclosed in the specification. Although each dependent claim listed below may directly depend on only one other claim, the disclosure of the possible implementations includes each dependent claim in combination with every other claim in the claim set.

While various actions are described as selecting, displaying, transferring, sending, receiving, generating, notifying, and storing, it will be understood that these example actions are occurring within an electronic computing and/or electronic networking environment and may require one or more computing devices, as described in FIG. 36, to complete such actions. Also, any annotations or markings generated on an image (in the above figures) may be of any color, including white, black, or another color.

No element, act, or instruction used in the present application should be construed as critical or essential unless explicitly described as such. Also, as used herein, the article “a” is intended to include one or more items and may be used interchangeably with “one or more.” Where only one item is intended, the term “one” or similar language is used. Further, the phrase “based on” is intended to mean “based, at least in part, on” unless explicitly stated otherwise.

In the preceding specification, various preferred embodiments have been described with reference to the accompanying drawings. It will, however, be evident that various modifications and changes may be made thereto, and additional embodiments may be implemented, without departing from the broader scope of the invention as set forth in the claims that follow. The specification and drawings are accordingly to be regarded in an illustrative rather than restrictive sense.

What is claimed is:

1. A method, comprising,
receiving, by a computing device, two-dimensional images, wherein the two-dimensional images are slices from three-dimensional electronic scans, and wherein the two-dimensional images are for a region of interest within a person's mouth; and
generating, by the computing device, a benchmark database;
wherein the benchmark database includes:
views of different mouth areas;
determining, by the computing device, a dental implant within the person's mouth.

2. The method of claim 1, wherein the received two-dimensional images is preprocessed data that has irrelevant electronic information removed.

3. The method of claim 1, wherein the different mount areas include full head views, medium views, and small views.

4. The method of claim 1, wherein the determining the dental implant location includes splitting the three-dimensional electronic scans.
